(19) United States
(12) Patent Application Publication
(10) Pub. No.: US 2022/0296763 A1
(43) Pub. Date: Sep. 22, 2022

(54) Personal Reusable Microdiffuser

(71) Applicants: Tina Margareta Powell, Bluffdale, UT (US); Therron Leon Powell, Bluffdale, UT (US)

(72) Inventors: Tina Margareta Powell, Bluffdale, UT (US); Therron Leon Powell, Bluffdale, UT (US)

(21) Appl. No.: 17/206,856

(22) Filed: Mar. 19, 2021

(51) Int. Cl. A61L 9/12; A61L 9/01

(52) U.S. Cl. CPC A61L 9/127; A61L 9/01; A61L 2209/15

(57) ABSTRACT

A small, reusable scent emitting device (FIG. 1 and FIG. 6), attachable to a variety of mask types (FIG. 5), with interchangeable reservoirs for use with multiple different scented liquids (2), connected to a flexible directional extremity trim able to needed length dictated by wearer use and mask type (FIG. 1, FIG. 4 and FIG. 6), and whose manufacture is distinct from any particular mask type.

### FEDERALLY SPONSORED RESEARCH

Not Applicable

### SEQUENCE LISTING OR PROGRAM

Not Applicable

### BACKGROUND

### Field of Invention

This invention relates to facial masks used for personal protection, and or aromatic or respiratory treatments, specifically to masks that have noxious or unpleasant odors associated with their ongoing use, environmental conditions or manufacturing processes which odors produce aversion to the user.

### Description of Prior Art

Facial masks have been used for centuries for their protective benefits, for the diffusion of life saving elements such as oxygen and as a medication route of administration for a variety of aesthetic and health care conditions. In recent history with the onset of COVID-19 masks are being recommended for long term use worldwide to deter the spread of the SARS-CoV-2 virus. Recommendations from health authorities have allowed for the use of a variety of masks of different materials and configurations. Common to most of these masks are odors that arise from the manufacturing process and from multiple uses. These odors can have noxious effects depending on the chemicals used in their manufacturing, the oral health of the user, the environment in which the masks are used, the number of uses, and the sensitivity of the user. Aversion to the accumulating odors emitted has a direct effect on the time a user can tolerate wearing the mask and the security with which the mask is fitted to the wearers face. Administered aromatic compounds have been shown to stimulate or support desirable biological, mental and emotional effects; addressing both body and mind, and can improve or relieve some of the unpleasant or undesirable aspects of wearing a mask.

Various methods have been designed in the past to provide a secure, more pleasant experience for mask users, that mitigate several of the inherent disadvantages of mask delivery systems of medical products or holistic, aromatherapy products. Irritations to the eyes and the skin of the wearer have been among the more uncomfortable events experienced. U.S. Pat. No. 9,629,401 B2 to Khalid Hassan al Malki (2017) addresses the eye irritation with deflectors attached to the mask that would block a portion of aerosolized particles from reaching the eyes of the mask wearer, while the medication or scented material inhaled by the wearer is provided by a removable cover and detachable medical strips. U.S. Pat. No. 4,503,851 to Klaus Braunroth (1985) addresses this issue with a masking attachment of absorbent material, saturated with an odor-masking substance that remained sealed on both sides. When odor-masking is desired, the seals are removed and the scent then disperses for a time. The mask is discarded after a single use.

U.S. Pat. No. 5,538,013 to Brannon (1996) describes a formed mask that includes an air-permeable body on the inner side of which is covered by an air permeable pocket or retaining clip, either or which allow for the use of interchangeable cartridges, saturated with various scents. This mask would allow for multiple uses. U.S. Pat. No. 5,636,628 to Dennis Barnum (1997) introduces a mask that has been chemically treated with a fixate carrier and one or more odor-masking agents in a volatile solvent. The fixate carrier is specified to mean an odor absorbing material, both the fixate and solvent materials depend on the odor requiring amelioration. U.S. Pat. No. 6,779,524 B2 to Strawder et al., (2004) presents a moldable, conformable mask with or without a scented icon that can be used during surgery or other procedures to deliver gasses to a patient before and after surgery/procedure. U.S. Pat. No. 4,790,307 to Haber et al., (1988) and U.S. Pat. No. 7,004,167 B2 to Yung-Chu Cheng (2006) describe a mask with a capsule enclosed between an inner and outer mask layer. Upon cracking the capsule the antibiotic and/or aromatic contents then diffuse into the mask for the benefit of the wearer.

The products represented in this list have a number of disadvantages. The solutions forwarded generally center on one particular type of mask and would not be equally effective if another mask were needed as a substitute. The shelf life and storage of the masks is unknown, as is the chemical stability of the odor ameliorators and absorbents. The availability of scents preferable to the wearer is not discussed. The permeability required to activate the suggested solutions does not discuss the possible compromise of protective aspects of the mask. Nor do they represent a refillable reservoir allowing selection and blending of fragrant or biological compounds.

### Objects and Advantages

The advantages of the personal, reusable micro-diffuser being proposed include but are not limited To:
(a) the ability to use this device on a variety of mask types.
(b) it can be attached to the outside or inside of a mask, right or left side, depending on the preference of the wearer.
(c) it has an adjustable emitter director that can be adapted to the mask and the face size of the wearer, allowing the emitter to more easily positioned relative to the wearer's nose and mouth; also decreasing the retraction of the mask against the face of the wearer with inhalation.
(d) it has the capability of attachment and removal from the mask without compromising the integrity of the mask.
(e) it is small and light weight, thus impacting minimally the gravitational and inertial degradation placed on the straps holding the mask securely to the wearers face.
(f) the emitter or wicking element can be adjusted to regulate the volatilization of scent to be noticeable to the wearer, but not so overwhelming as to negatively impact the wearers or others within the immediate vicinity of the wearer.
(g) the chance of the scented liquid coming into direct contact with the skin of the mask wearer is very small if the device is attached as recommended.
(h) the possible number of scented liquids available to the user is almost unlimited by the device and at the discretion of the user's preference.
(i) the transparent nature of the reservoir, in the primary configuration allows the wearer to see when a refill is necessary.
(j) manufacture of the device is separate from the mask.

Further objects and advantages of the invention will become apparent from a consideration of the drawings and ensuing description.

### SUMMARY

The Personal Reusable Micro-diffuser provides mask wearers a pleasant alternative to the noxious odors associated with mask wearing caused by mask manufacturing processes, repeated mask use, oral health of the mask wearer, or environmental emissions. It is light weight and can be attached to a variety of masks. It allows for the use of scents preferred by the mask wearer that can be changed as desired. It is discreet either inside or outside the mask, both in appearance and in the strength of content emission.

### DRAWINGS

Drawing Figures

In the drawings, closely related figures have the same number, but different alphabetic suffixes.

FIG. 1 (1a-1b) shows the primary configurations of the micro-diffuser (a) without an attachment device, (b) with a pin back, secured with heat shrink.

Reservoirs, Various Types show the reservoirs in the primary configuration along with others in alternative configurations of the diffuser.

Attachments, Various Types shows the attachment devices used in both the primary and alternative configurations, used in combination with the pin or clip (11a and 11B) and the heat shrink (10).

Various Parts shows directional extremities (9a and 9b) PTFE tubing and small gauge wire used to guide the wick to the optimal placement for the wearer, different wick types (12a-12c) fiber, sticks and thread. Shown in this drawing are also hook and loop self-adhesive circles and a threader, while not part of the diffuser they are extremely helpful with holding the emitter in place on seamless/molded masks, inserting the wick into the tubing and controlling the curvature of the tubing in tight spaces decreasing misdirection, kinking, and cracking of the tubing.

FIG. 2 (2a-2f) shows variation possibilities of mask types, (a) paper, (b) cloth, (c) molded (d) mask filter, (e-f) CPAP and the in-line (Variations)

FIG. 3 (3a-3c) shows possible Alternate Configurations

### REFERENCE NUMERALS IN DRAWINGS

7—Reservoir
8—Cap
9—Directing Extremity (tubing/wire; flexible/rigid)
10—Heat shrink and other binding materials
11—Attachment devices (pin and clip)
12—Adjustable essence emitter/wick

### DETAILED DESCRIPTION

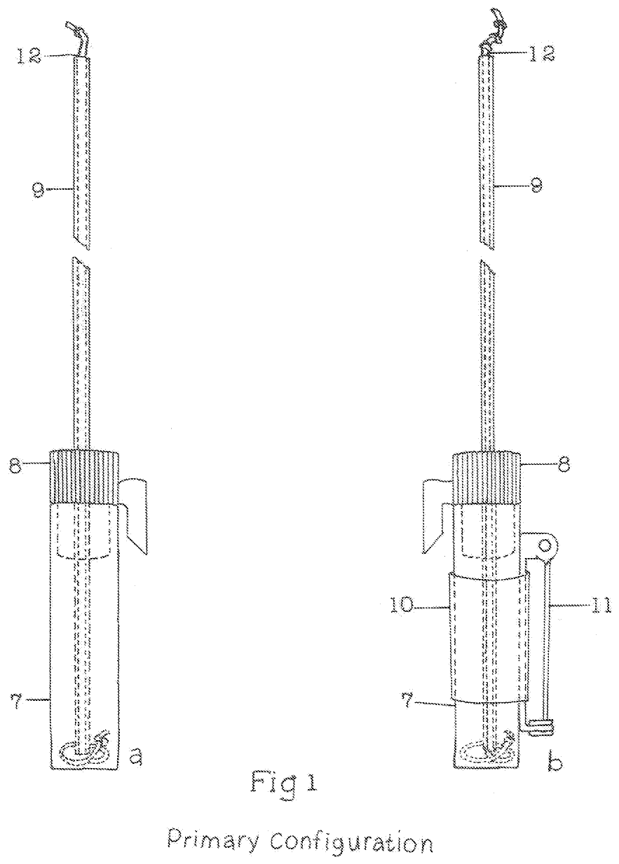

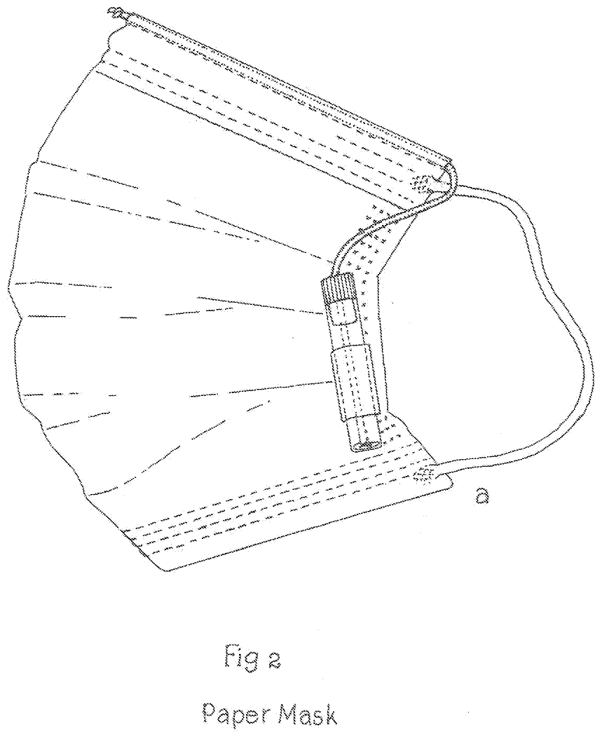

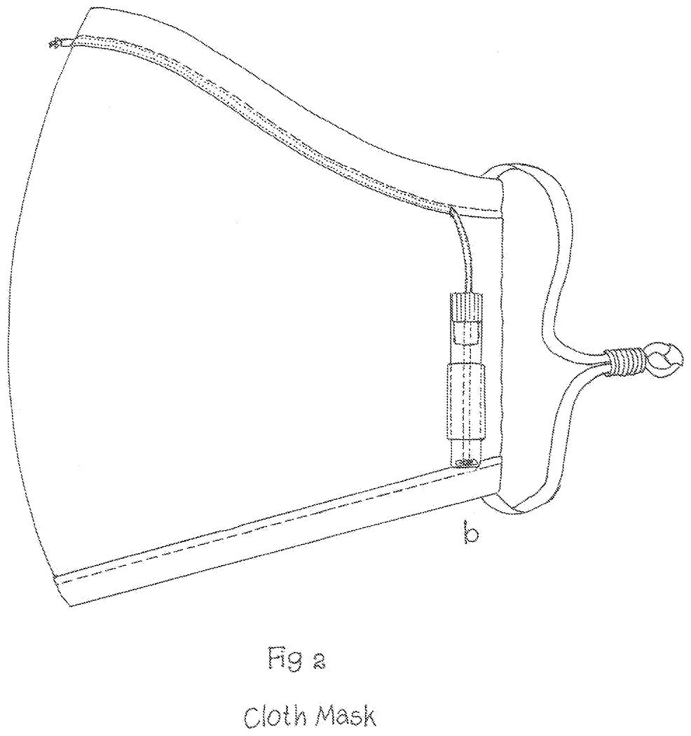

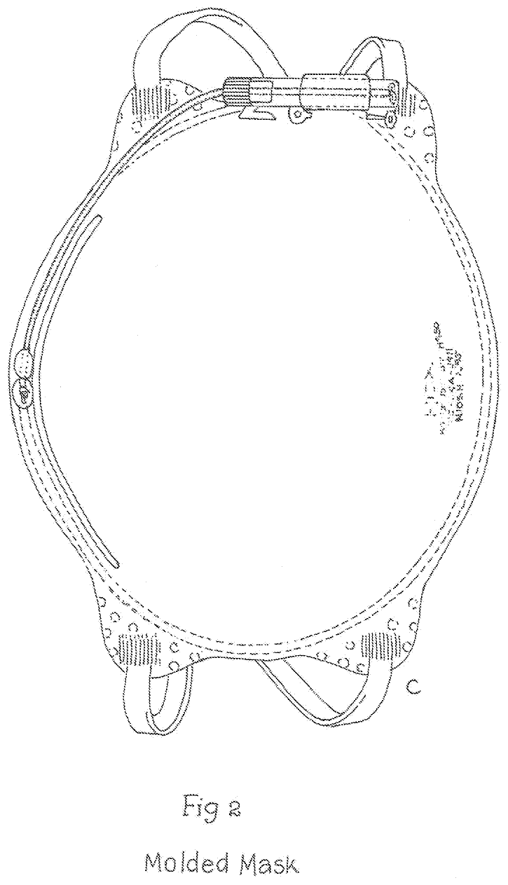

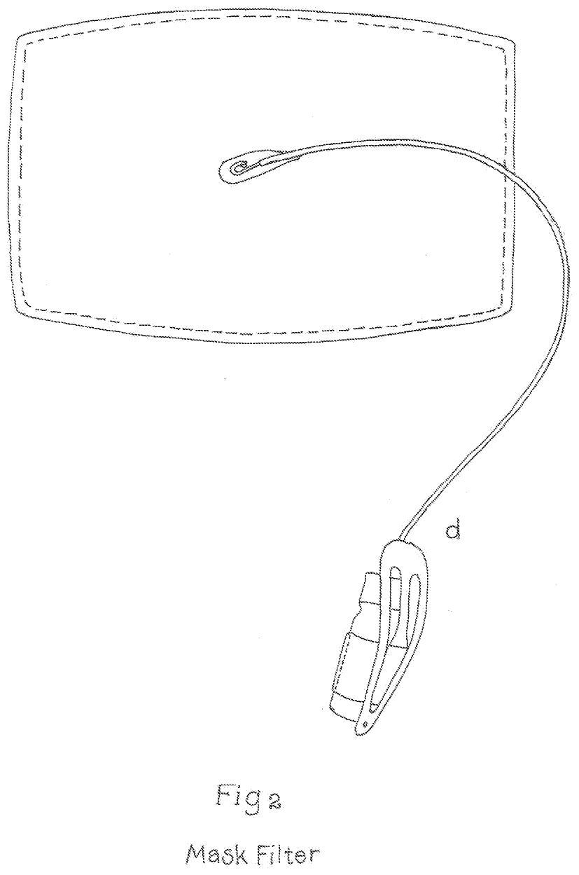

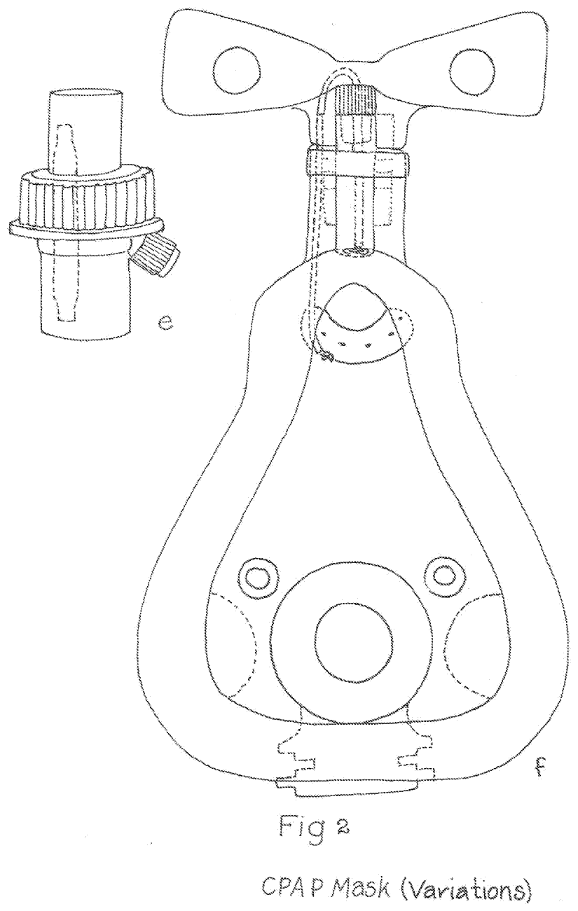

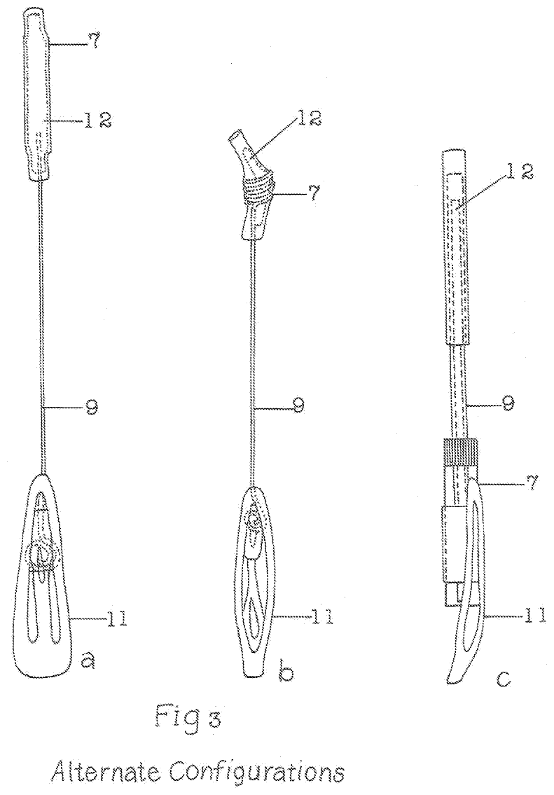

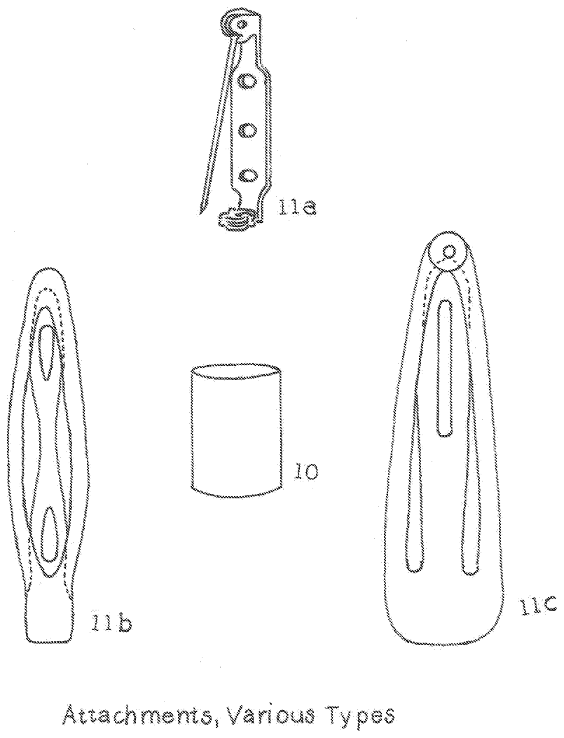

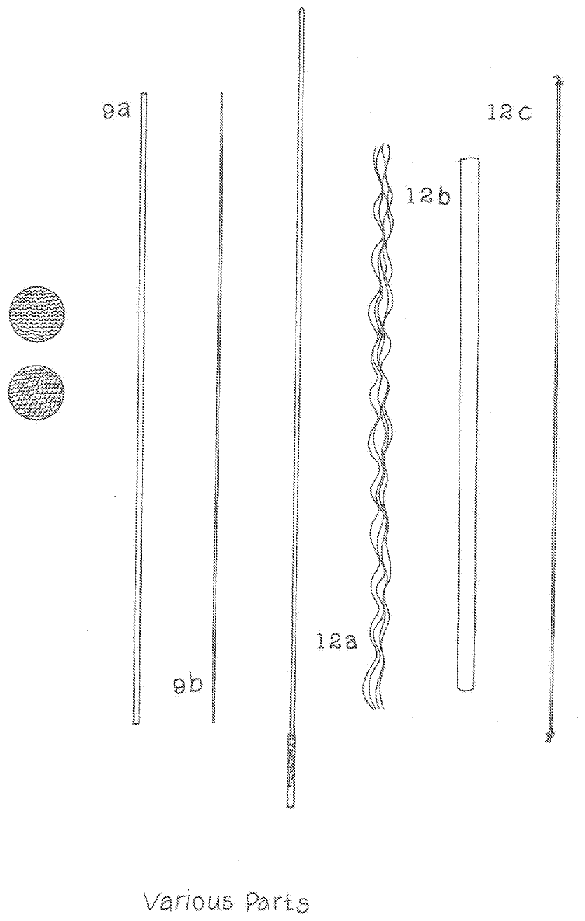

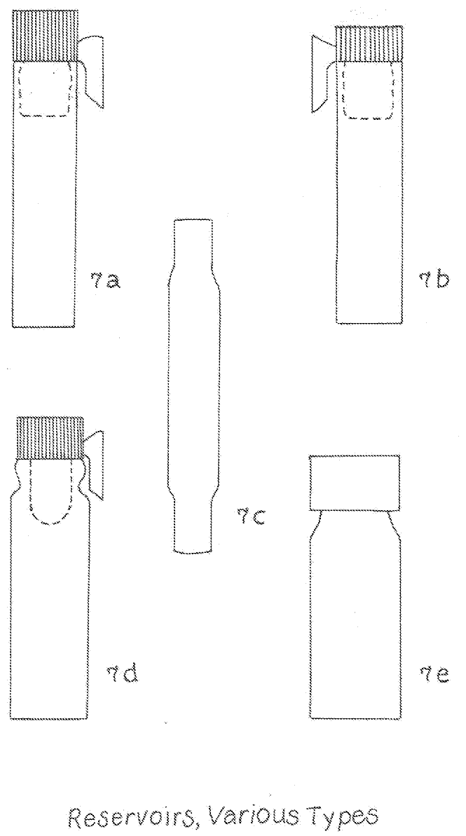

The Personal Reusable Micro-diffuser incorporates an impermeable reservoir of glass or PTFE, (FIG. 1 and Reservoirs, Various Types) fitted, if required, with a tight fitting cap. Out of the center of the cap extending on one or either side is a directional guiding device (FIG. 1 and Various Parts). An adhesive or sealant is placed around the tubing to secure it to the cap and impede leakage of liquid, (not shown). This guiding device is made of PTFE tubing, fiber sticks, or small gauge wire (FIG. 1 and Various Parts). The length of this guiding device can be altered to fit the needs of the mask and wearers face. In the primary configuration which comprises the PTFE tubing, a fibrous wicking material transmits and emits the scent. Knots tied at both ends of this wick after insertion prevent the wick from coming out of the tubing and help control emission of scent. The attachment device for the primary configuration is a pin back secured with heat shrink. A circle of hook and loop fastener (self-adhesive) (Various Parts), can be placed on either side of the tubing near the wick ending to attach it to the mask near the bridge of the nose (FIG. 2c) or other desired location. The reservoir can be attached to either the right or left side of the mask by way of the pin or clip banded to the reservoir. Alternatively it can be slid into a pocket if part of the mask construction, or a fabric loop/tag on either side of the mask if available (not shown in drawings). Other configurations are also possible (FIGS. 3a-3c).

Operation (FIGS. 1a-1b, 2a-2f, 3a-3c)

On the initial use of the personal, reusable, micro-diffuser, the tube containing the wick is placed so that the distal end of the tube from the reservoir is near the wearer's nose. This is accomplished by threading the tube through a channel formed naturally in the seams of the mask during the manufacture. The wicking material can be inserted into the tube before or after the tube is in place. It should be knotted at both ends after insertion. The size and number of knots, as well as the distance the wick protrudes from the end of the tubing will affect the amount of scent emitted from the diffuser (Various Parts). Fill the reservoir with the desired liquid and press the cap on tightly (FIG. 1a-1b). Attach the reservoir to the mask with the pin, (FIG. 2a-2f) or deposit it into a pocket or loop if present on the side of the mask near the tube insertion point. Wear the mask as recommended. Alternate configurations (FIG. 3a-3c) can be clipped to either side of the mask and the emitter can be guide to the most convenient place inside the mask for the wearer or on to a respiration device or in-line supply tubing.

### CONCLUSION

Thus the reader will see that the Personal Reusable Micro-diffuser provides a versatile, light weight, attachable/interchangeable device that can be used with a variety of mask or breathing apparatus types and scent options, without compromising the integrity or function of the breathing apparatus.

While my above description contains many specificities, these should not be construed as the limitations on the scope of the invention, but rather as an exemplification of one preferred embodiment thereof. Many other variations are possible, as suggested in the ramifications.

Accordingly, the scope of the invention should be determined not by the embodiment(s) illustrated, but by the appended claims and their legal equivalents

1. A small scent emitting instrument comprising:
(a) An interchangeable reservoir to hold a plurality of scented liquids
(b) A flexible, directional extremity protruding from either or both sides of said reservoir, containing a fiber wick
the flexible, directional extremity of claim b allows for emission control
(c) A method of allowing said instrument to attach to a variety of masks
Whereby said instrument provides various pleasant aromas for extended periods of said mask wearing, thus decreasing aversion to the wearing of beneficial facial coverings.
